(19) United States
(12) Patent Application Publication
(10) Pub. No.: US 2005/0074902 A1
(43) Pub. Date: Apr. 7, 2005

(54) Method for obtaining a unique calibration system applied to multi-parametric doses of biological samples, immunologicaLreagent prepared for that purpose, and dosage method

(75) Inventor: Pascale Laroche, Paris (FR)

Correspondence Address: YOUNG & THOMPSON, 745 SOUTH 23RD STREET, 2ND FLOOR, ARLINGTON, VA, 22202, US

(73) Assignee: Biomedical Diagnostics SA, 77183 Croissy Beaubourg (FR)

(21) Appl. No.: 10/500,563

(22) PCT Filed: Jan. 10, 2003
(86) PCT No.: PCT/FR03/00069

(30) Foreign Application Priority Data
Jan. 11, 2002 (FR) 02/00294

(51) Int. Cl. G01N 33/543

(52) U.S. Cl. 436/518

(57) ABSTRACT

Different categories of particles are used, each of which is sensitized by a specific ligand for one of the analytes to be dosed. An immunological reagent consisting of a mixture of each of the particle categories having been sensitized by a specific quantity of the respective ligand is prepared. The signals resulting from the interaction between the immunological reagent and the biological sample on one hand and a calibration standard on the other are then measured. A correction factor is subsequently determined which is applied to the different resulting signals in such a way that each analyte of the sample can be titrated in biological units.

The present invention relates in general to the field of analyte assays using biological samples, and more particularly to the calibration of such assaying methods. Specifically, a subject of the present invention is a novel method for obtaining a single calibration system for simultaneously quantifying several analytes, whatever the number thereof, and in the same reaction medium.

The expression “single” calibration system is intended to mean a sole system whatever the number of analytes simultaneously investigated.

Since the quantification of analytes included in a biological sample has become essential both in terms of research and in terms of conventional medicine, such quantification has been the subject of many developments aimed not only at improving the reliability and reproducibility of assays, but also at decreasing the costs thereof.

Conventional techniques consist in assaying each analyte separately and, to do this, in performing, for each of said analytes, a calibration specific to the system used. This results in a large number of manipulations and a considerable consumption of reagents, the consequence of which is a relatively high cost.

While operating in such a manner remains conceivable in the context of research, where the quantity of parameters to be assayed is limited and targeted, it imposes a serious cost problem in the context of medicine, where, in test laboratories, the rapid assaying of a large number of analytes is sought.

A technique for quantifying, in a precise and reproducible manner, several analytes in one and the same assay using the same sample has recently been described, in particular in international patent applications WO 99/36564 and WO 97/14028. This technique is based on the use of various categories of fluorescent particles, each category of particles being sensitized with ligands having different properties, said ligands being appropriate for binding directly or indirectly to the analytes and generally consisting of antibodies or antigens. The reaction between ligand and analytes is quantified by means of a fluorescent compound having excitation and emission wavelengths different from those of the fluorescent particles.

Thus, simple reading by flow cytometry makes it possible to quantify the reaction associated with each category of particles and therefore to deduce from said quantification the amounts of the various analytes present in the same sample.

However, this quantification requires the prior construction of as many calibration systems as there are analytes to be assayed, i.e. performing X*Y assays reserved for calibration, X being the number of analytes to be simultaneously assayed (or the number of particles sensitized with ligands having different properties) and Y being the number of calibration points. This method therefore not only results in increased handling, but also a problem of practical management, due to the overloading of the microtitration plate wells (the assays generally being carried out on 96-well ELISA microtitration plates), and also an increase in the handling time.

For example, for the simultaneous assaying of 10 analytes, this system involves constituting 10 different calibration systems, i.e. for 100 samples tested, an additional consumption of reagents ranging from 10 to 40% depending on the number of calibration points, and an obvious problem of overloading.

The present invention aims to remedy the disadvantages of the prior art by proposing a novel single, rapid, simple and reproducible calibration method which makes it possible to decrease the consumption of reagents dedicated to the calibration, while at the same time also decreasing the overloading of the titration plates.

This aim is achieved in the sense that the present invention relates to a calibration method requiring only a single calibration system, for simultaneously quantifying several analytes in the same biological sample, which method is based on the preparation and use of a specific immunoreagent.

More particularly, a first aspect of the present invention relates to a method for preparing an immunoreagent which goes into making up a calibration system applied to the assaying of multiple analytes in the same biological sample, said method using various categories of particles, each category of particles being sensitized by association with a ligand specific for one of the analytes to be assayed, said method comprising the steps which consist in:
a) determining, for each category of sensitized particles and for each of n given amounts of ligand associated with said particles, the curve of response as a function of the concentration of homologous compound, over a range of concentrations corresponding to the known measurement range of the analyte to be assayed;
b) selecting, for each category of sensitized particles, the curve corresponding to the smallest amount of ligand which gives a significant response signal and which is compatible with the use of a sample dilution and of a labeling reagent, which are common to all the analytes simultaneously assayed;
c) evaluating, for each category of particles sensitized, according to the curve selected in step b), the mean signal corresponding to the signal associated with a point characteristic of each of said curves, thus obtaining as many mean signals as there are categories of particles;
d) adjusting, where appropriate, the amounts of ligand associated with each category of particles such that all the mean signals evaluated in step c) are within a ratio of 1 to 5, and
e) mixing, in an appropriate solvent, the various categories of sensitized particles which correspond to the criterion of step d).

The method which is the subject of the present invention therefore applies to assaying methods using various categories of sensitized particles. More particularly, such methods consist mainly of immunoassays of the following type:
direct assaying, by an immunometric or “sandwich” method, for example, of an antibody which is reacted with the specific antigen attached beforehand to the particles,
direct assaying, by an immunometric or “sandwich” method, for example, of an antigen which is reacted with the specific antibody attached beforehand to the particles, or
indirect assaying, by a “competition” method, of an antigen which competes with a labeled similar antigen for its reaction with the antibody attached beforehand to the particles, or vice versa.

These various assays will be described subsequently in greater detail.

The reaction developed by bringing the various categories of particles, each sensitized with a ligand, into contact with the analytes to be assayed is measured by flow cytometry according to various particle identification modes.

Mention may be made, for example, of particles of different colors and of uniform size (identified according to their specific color, with quantification of the analyte-ligand reactions by addition of an external compound having excitation and emission wavelengths different from those of the particles), of particles of different sizes and of identical color (identified according to their size, with quantification of the analyte-ligand reactions by measurement of the size of the resulting aggregates) or else of particles of different sizes and of identical color (classified according to their size, with quantification of the analyte-ligand reactions by addition of an external compound having excitation and emission wavelengths different from those of the particles). In practice, one or other of these methods is selected according to the more or less large number of parameters to be tested.

These particles generally consist of polystyrene. However, they may also consist of any polymer which confers on them specific physicochemical properties (functionality, density or alternatively solubility), such as styrene-maleic acid, styrene-methacrylic acid, styrene-acrylamide or poly(methyl methacrylate).

The term “ligand” should be understood to mean an antigen, a monoclonal or polyclonal antibody, or any molecule capable of attaching to the particles and which is capable of interacting, in any manner whatsoever, directly or indirectly, with the analyte to be assayed.

The term “antigen” is intended to mean any substance from which the production of antibodies can be generated. Among these substances, mention may be made of proteins, haptens, allergens, peptides, medicinal products or drugs.

The term “homologous compound” should be understood to mean an antibody, an antigen or any molecule capable of binding to a given ligand, and for which the site of interaction with said ligand is similar to the site of interaction between the analyte to be assayed and said ligand. The homologous compound can be the analyte itself.

The first step of the method therefore consists in determining, for each category of sensitized particles, as described above, the correlation which exists, for a given amount of ligand attached to the particles, between the amount of homologous compound and the size of the signal emitted, representative of the ligand/analyte interactions.

In practice, for each category of particles, several trials are carried out by attaching a given amount, different for each trial, of the same ligand to said particles, then bringing the particles thus sensitized into contact with various amounts of homologous compound, identical for all the trials, and measuring the signal resulting from their interaction.

More particularly, the amounts of ligand used vary by steps of 2 to 4.

The various amounts of homologous compound are selected so as to be included within the measurement range for the analyte intended to be assayed. Thus, if the intention is to assay an analyte, the concentration of which can range from 0 to 100 biological units, six given amounts of homologous compound will, for example, be chosen, corresponding to 0, 20, 40, 60, 80 and 100 biological units. In practice, a population of samples is used which are characterized and certified with respect to clinical biological criteria, i.e. for which the presence or absence of a given analyte is validated by a clinical diagnosis and/or by estimation of the analyte using one or more other assaying method(s).

The result of this operation is that a dose/response curve is obtained between the amount of homologous compound and the signals obtained for each of the n given amounts of ligand.

The idea of expressing the concentration in biological units is based on allocating the measurement range for each of the analytes to be assayed a range of 0-150, for example. It is understood that these measurement ranges differ, in the absolute, from one analyte to another and that, consequently, the same concentration of various analytes expressed in biological units, for example 95 biological units (abbreviated to 95 BU), does not correspond to the same concentration, in the absolute, of these same analytes. The concentration of homologous compounds is expressed, in the same way, in biological units over an identical range for all the analytes.

In this respect, it should be noted that the sensitization of the particles can be carried out in various ways, namely by covalence, by means of a biologically and/or chemically reactive intermediate molecular layer, or by using an affinity-based interaction system.

The sensitized particles, insofar as they comprise a reactive functionality, can be sensitized by covalence. Thus, many immobilization protocols have been described as involving, with no intended limitation, the following functional groups between the particles and the ligand:
COOH/R—NH₂ or R—OH,
CO—NH₂/NH₂—NH₂,
C(═O)—O—CH₃/NH₂—NH₂,
NH₂CHO/RCOOH, or alternatively
R—NH₂ or NH₂—NH₂.

It should be noted that, when most of the couples mentioned above are used, it is necessary to pre-activate certain functions, such as for example, the COOH functions, in order for them to be able to react, for example, with the functional groups of the ligand, such as aliphatic or aromatic amine groups.

According to another preferred embodiment, the activation can be carried out with water-soluble carbodiimides, and can be performed in a single step or, according to a particularly preferred embodiment, in two steps, with the carbodiimide alone or in the presence of N-hydroxysuccinimide. It is followed by elimination of the excess activator before attachment of the ligand.

According to yet another embodiment, a homo- or heterobifunctional spacer arm, having reactive chemical functions at each end, which are respectively identical or different, can be attached to the pre-activated particles. These arms, of varying length and functionality, can be activated and coupled by covalence, in a first step, to the particles by one of the ends and, in a second step, to the compound to be attached, via the other end.

Chemically or biologically active intermediates can also be used for the sensitization. They create, at the surface of the microparticles, an intermediate molecular layer consisting of a protein (such as albumin), a mixture of proteins or a polymer (such as polylysine). This method is particularly used in the case of small particles which can then be coupled, by means of linking agents, to the functions of this intermediate layer.

Another route of sensitization, this time noncovalent, consists in using, with no intended limitation, affinity-based interaction systems:
the biotin-avidin system, using particles coupled to avidin which are brought into contact with compounds which have been biotinylated beforehand,
protein A or protein G, having available a receptor for the immunoglobulin Fc fragment, making it possible to immobilize an antibody and to direct its Fab fragments, which are responsible for antigenic recognition, outward, or alternatively
indirect immobilization by means of an antibody specific for the compound to be attached.

This list is in no way limiting and it is obvious that any similar method of sensitization known to those skilled in the art can also be used.

For each ligand to be attached, the selection of one of the abovementioned systems is initially done during the initial phase of setting up. The operating conditions are then fixed and subsequently applied systematically.

In a preferred embodiment, the sensitization medium consists of buffered water having a pH of between 5 and 9 and an ionic strength of between 0.01 and 0.1. In order to keep the medium constant, the ligand can be suspended in the same medium before performing said sensitization.

The advantage of such a system lies in its simplicity of use, its low cost and its reactional independence with respect to the other simultaneous reactions. That is made possible by the fact that the reaction produced by each category of sensitized particles is standardized and reproducible, that the signal provided by this reaction is representative of all the ligand-analyte reactions developing simultaneously, and that all these reactions are controlled at the level of the method of sensitization for each category of particles in such a way that they all produce a similar signal in the presence of samples having the same degree of positivity, i.e. comprising substantially the same amount of the analyte assayed, as expressed over its specific range of biological units.

Once the dose/response curves have been obtained, the curve corresponding to the smallest amount of ligand giving, graphically, a reading signal that is significant and compatible with the use of a common sample dilution and a common labeling reagent for all the analytes simultaneously detected is determined, for each category of sensitized particles, as described in step b).

The expression “significant response signal” should be understood to mean a signal which is sufficiently high to ensure reading precision over the entire measurement range.

In practice, the curve selected should be substantially linear and should have a sufficiently high measurement value.

Each type of particle is therefore sensitized by one of the techniques described above, using the amount of ligand determined above.

The subsequent step c) then consists in determining the mean signal (MS) associated with a concentration of biological units, identical for all the analytes.

To do this, a point characteristic of the dose/response curve selected in step b) is selected and the signal corresponding graphically to said point is taken as mean signal MS.

The characteristic point, also called positive point or high positivity point, is chosen arbitrarily within the upper quarter of the linear part of the curve.

The means signals MS obtained for the various categories of sensitized particles are then compared with one another and, if necessary, adjusted such that they are all within a ratio of 1 to 5. In practice, for the categories of particles having an MS not within said ratio, the reactivity is then readjusted by selecting, from the dose/response curve, another amount of ligand and recommencing the operation of sensitizing said particles with this new amount of ligand until the resulting MS falls within the abovementioned ratio of 1 to 5.

The fact that the MS values for the various sensitized particles constituting the immunoreagent are all within a ratio of 1 to 5 is the determining factor in ensuring relatively equivalent immunoreactivity for each type of sensitized particles used and therefore in obtaining, within a measurement range compatible with the biological reality, values that are sufficiently close to allow a single calibration system common to all the analytes to be used.

The immunoreagent is then made up by mixing the various types of sensitized particles thus obtained, in a solvent.

As possible solvents, mention may be made of: mixtures of proteins and amino acids or alternatively a mixture of proteins that are inert with respect to the analytes to be assayed and allow particle stabilization.

In any event, the solvents used are selected in such a way as to conserve all the sensitized particles, and their choice is therefore dictated by the nature of the most restrictive ligands.

As a result, said solvent can be defined such that it is suitable for and common to all the ligands.

In order to make such a method for preparing the immunoreagent, involving the individual reactivity of various particles coupled to specific ligands, industrially applicable, the experimental conditions for sensitization, namely the pH, the ionic strength, the volume, the temperature or the duration of coupling, are preset and standardized for all the particles. Only the amount of ligand to be adjusted subsequently varies, as described above.

In practice, the solvent used is buffered at a pH of between 7 and 9 and has an ionic strength of between 0.01 and 0.1.

According to a second aspect of the present invention, it relates to an immunoreagent intended for the assaying of multiple analytes in biological samples, said reagent comprising a solvent and, mixed in with said solvent, various categories of particles, each of which is sensitized by association with a given amount of a ligand specific for one of the analytes to be assayed, with, for each of the categories of particles, and for a given concentration of compound homologous to the ligand, as expressed in biological units, said given amount of ligand resulting in a signal referred to as “mean signal”, which is within a ratio of 1 to 5 with the mean signals obtained for the other categories of particles.

According to a third aspect, the present invention relates to a kit for assaying multiple analytes in biological samples using various categories of particles, each category of particles being sensitized with a ligand specific for one of the analytes to be assayed, which comprises:
i) an immunoreagent derived from the method as described above,
ii) at least one calibration standard consisting of a single homologous compound which reacts with one of the categories of particles which goes into making up the immunoreagent,
iii) a table of correspondence between the concentration, expressed in biological units, of the homologous compound constituting the calibration standard and that of each of the compounds homologous to the other ligands attached to the other categories of particles which go into making up the immunoreagent, and
iv) a labeling reagent.

More particularly, said calibration standard comprises a homologous compound which reacts directly or indirectly with the ligand attached to the sensitized particle.

The indirect attachment can be carried out, for example, by means of a protein used to saturate the sites of attachment on the particle which are not occupied by the ligand. This protein should be chosen so as not to interfere with the specific system and should be present on a single category of particles. In this case, the quantification medium will have to contain a labeling reagent specific for the analytes to be assayed (consisting of one or more compounds) and a labeling reagent specific for the inert protein.

In a first embodiment, said homologous compound is of the same origin as the analyte to be assayed, for example of human origin.

In a second embodiment, said homologous compound and the analyte to be assayed are of different origins.

While, for example, the analyte to be assayed is of human origin, such as autoantibodies present in human blood samples, the homologous compound may be, for example, of animal origin, provided that it is capable of reacting specifically with the ligand. The quantification medium will, in this case, have to contain both a labeling reagent specific for the samples of human origin and a labeling reagent specific for the standard of animal origin.

The preparation of the calibration standard involves prior optimization of its concentration so as to produce an MS within a ratio of 1 to 5 with respect to the MS values obtained for each category of particles sensitized during the preparation of the immunoreagent.

As regards the “correspondence table”, it consists of a reference system, which may be in the form of a table, a a graph, etc., which gives the various concentrations, expressed in biological units, of the homologous compound constituting the calibration standard and that of each of the compounds homologous to the other ligands attached to the other categories of particles which go into making up the immunoreagent.

The correspondence table is produced empirically, prior to the preparation of the kits, in the following way.

Several samples, comprising different and known amounts of analytes to be assayed, are reacted with the immunoreagent which will be part of the kit. The resulting signals are measured and, for each analyte, the linear regression line connecting the emitted signals to the known amounts, expressed in biological units, of the assayed analytes, is plotted according to an equation of y=ax type, with y=signal and x=concentration of biological units.

Similarly, the signal obtained during the reaction between the calibration standard and the immunoreagent is measured and the concentration, expressed in biological units, corresponding to this same signal value is noted on each curve previously obtained.

All these analyte concentrations, expressed in biological units, constitute the correspondence table.

The concentration of the standard can then be verified from the correspondence table and adjusted if necessary.

In one variant, it may be desired to produce a calibration range, i.e. using several calibration standards each consisting of the same homologous compound, but according to various concentrations.

In this case, the principle for producing the correspondence table remains the same, apart from the fact that, for each analyte, using the various signals obtained, the regression line connecting the emitted signals with the known amounts, in biological units, of the analytes to be assayed is plotted according to an equation of log y=a(log x) type, y=signal and x=concentration in biological units.

This equation is then applied to the signals corresponding to the various known concentrations of calibration standard so as to deduce the correspondence between each of said concentrations and the concentration of each analyte, expressed in biological units.

As regards the “labeling reagent”, it consists of an immunocompound capable of quantifying the reaction between the analytes and the immunoreagent, said immunocompound being coupled to a label which is preferably a fluorochrome.

The term “immunocompound” should be understood to mean any natural or synthetic compound capable of forming a complex specifically with a complementary compound, such as antibodies and antigens.

According to a preferred embodiment, said labeling reagent reacts with the homologous compound or analyte to be assayed/ligand complex, in which case the assaying is direct, or with the uncomplexed ligand, in which case the assaying is indirect.

In addition, the labeling reagent can be prepared from a single compound, which reacts with, for example, an antigenic specificity common to all the homologous compounds, or can be the result of mixing various compounds which react specifically with various homologous compounds.

According to a final aspect of the present invention, it relates to a method for assaying analytes present in a biological sample, and more particularly to a method for using the kit as described above, which consists in measuring the signals resulting from the interaction between the immunoreagent and, firstly, the biological sample, and secondly, the calibration standard, and in determining and applying, to the various resulting signals, a correction factor so as to obtain the titration, expressed in biological units, of each analyte of the sample, said correction factor being the ratio between the signal obtained for the calibration standard and the concentrations derived from the correspondence table.

The method for using the kit according to the present invention is therefore based on three steps, namely a first step of determination of the various emitted signals by flow cytometry, a second step of calculation so as to determine the various correction factors from the signal emitted by the calibration standard, and a third step of calculation for the titration of each analyte of the sample based on the correction factor previously obtained and the signals emitted by the various samples tested.

More particularly, the method which is the subject of the present invention comprises the following steps:
a) incubating, firstly, the biological sample and, secondly, the calibration standard with a predetermined amount of immunoreagent;
b) adding the labeling reagent;
c) measuring, by flow cytometry, the signals emitted, firstly, by the calibration standard and secondly, by the sample;
d) determining, for each category of particles sensitized, a correction factor which corresponds to the ratio between the concentration in biological units of each analyte to be assayed as given by the correspondence table and the signal corresponding to the calibration standard and measured in step c);
e) multiplying the signal emitted by each category of particles and measured in step c) by said correction factor, calculated for each analyte, so as to deduce therefrom the concentration in biological units of each analyte in the sample tested.

In a first variant, the method which is the subject of the present invention can be applied to direct assays. In such assays, the labeling reagent is specific for the analytes intended to be assayed and, as a result, the amount of analytes included in the sample is directly proportional to the signal emitted by said labeling reagent.

A first application to the simultaneous and direct quantification of antibodies having different antigenic specificities is envisioned with said analytes to be assayed which consist of antibodies, said ligands which consist of antigens and said labeling reagent consisting of one or more second antibodies labeled with a fluorochrome which react specifically with the antibodies intended to be assayed.

In this case, the immunoreagent consists of various categories of particles, each sensitized with a specific antigen. The antibodies intended to be assayed will form complexes with said antigen(s) attached to the particles. The labeling reagent, which consists of one or more second antibody or antibodies labeled with a fluorochrome and specific for the antibodies intended to be assayed, will bind to said antibodies precomplexed with the antigens forming the layer sensitizing the particles. Thus, the signal emitted by the fluorochrome associated with each category of particles is proportional to the amount of antibodies present in the sample.

The term “second antibodies” should be understood to mean any substance capable of forming complexes with the antibodies intended to be assayed, i.e. capable of reacting with an epitope of said antibodies which is different from that used to provide the binding thereof to the ligand.

A second application to the simultaneous and direct quantification of various antigens is envisioned with said analytes to be assayed which consist of antigens, said ligands which consist of antibodies and said labeling reagent which consists of a mixture of second antibodies labeled with a fluorochrome which react specifically with the antigens intended to be assayed.

The principle here is identical to that described above.

In a second variant, the method which is the subject of the present invention can be applied to “indirect” assays. Such assays are no longer based solely on the complementarity between two immunological species, but on the competition, between two similar immunological species, for forming complexes with a third immunological species.

Thus, according to a first possibility, the simultaneous and indirect quantification of various antigens with said analytes to be assayed which consist of antigens, said ligands which consist of antibodies and said labeling reagent which consists of a mixture of antigens labeled with a fluorochrome which compete with the analytes to be assayed for forming complexes with the ligands is envisioned.

In this case, the immunoreagent consists of various categories of particles, each sensitized with a specific antibody. The antigens intended to be assayed will then compete with said antigen(s), labeled with a fluorochrome, constituting the labeling reagent. Thus, an increase in the concentration of antigen intended to be assayed results in an increase in the concentration of antigen intended to be assayed/bound antibody complexes, to the detriment of the formation of labeled antigen/bound antibody complexes, with a correlating decrease in the signal. As a result of this, the signal emitted by the fluorochrome associated with each category of particles is inversely proportional to the amount of antigens present in the sample.

A second application to the simultaneous and indirect quantification of various antibodies is envisioned with said analytes to be assayed which consist of antibodies, said ligands which consist of antigens and said labeling reagent which consists of a mixture of antibodies labeled with a fluorochrome which compete with the analytes to be assayed for forming complexes with the ligands.

The present invention will be understood more thoroughly on reading the following examples.

Materials and Methods

The system of sensitized particles used in the examples below belongs to the Luminex® system.

The particles used are uniform in size (5.5 μm) and differ from one another by virtue of a specific coloration (100 colors, from red to orange). The system for detecting these particles is a flow cytometer interfaced with a computer system for processing the signal emitted with various lasers. A first laser, by classifying each category of particles on the basis of its unique fluorescence intensity, makes it possible to identify the compound which is analyzed. In parallel, a green laser excites an external fluorescent compound used to quantify the reaction specifically associated with each category of colored particles.

According to other embodiments, and according to the knowledge of those skilled in the art, lasers other than a green laser may also be used.

### EXAMPLE 1

### Simultaneous Assaying of the Antinuclear Antibodies (ANA) Directed Against the Following Antigens: SSA, SSB, Sm, Sm/RNP, Scl70, Jo1, dsDNA, Centromere

a) Preparation of the Immunoreagent.

Eight categories of colored polystyrene particles functionalized with COOH groups are selected. Prior to sensitization, the particles are activated by means of a method which consists in:
separating the eight categories of particles in eight different tubes,
adding 1 ml of a freshly prepared solution of carbodiimide, at between 10 and 40 mg/ml, to 1 ml of each type of particles (i.e. approximately 107 particles),
incubating for 20 to 60 minutes at ambient temperature, and
washing the particles three times with distilled water, centrifuging at 10 000 g for 2 minutes to remove the excess carbodiimide.

In parallel, the amounts of each ligand, or antigen, which had to be attached to the various categories of particles were determined according to the invention, as described above.

The particles thus activated are then sensitized with different antigens, according to the category of particles, by suspending each solution of activated particles in a volume of 1 ml containing from 50 to 500 μg of different antigens.

The system below is thus obtained:

| Category of particles | Nature of the antigen (ligand) |
|---|---|
| 1 | SSA |
| 2 | SSB |
| 3 | Sm |
| 4 | Sm/NRP |
| 5 | Scl70 |
| 6 | Jo1 |
| 7 | dsDNA |
| 8 | centromere |

The following steps consist in:
incubating for 4 to 6 hours at ambient temperature,
washing the particles with distilled water,
suspending it in a buffer, pH 8, containing a mixture of amino substances in order to saturate the free sites and return the particles to a concentration of the order of 10⁶ to 5×10⁶ particles per ml.

The immunoreagent then consists of a mixture of an aliquot of 500 μl of each category of colored and sensitized particles, and it is stored at +4° C. until it is used.

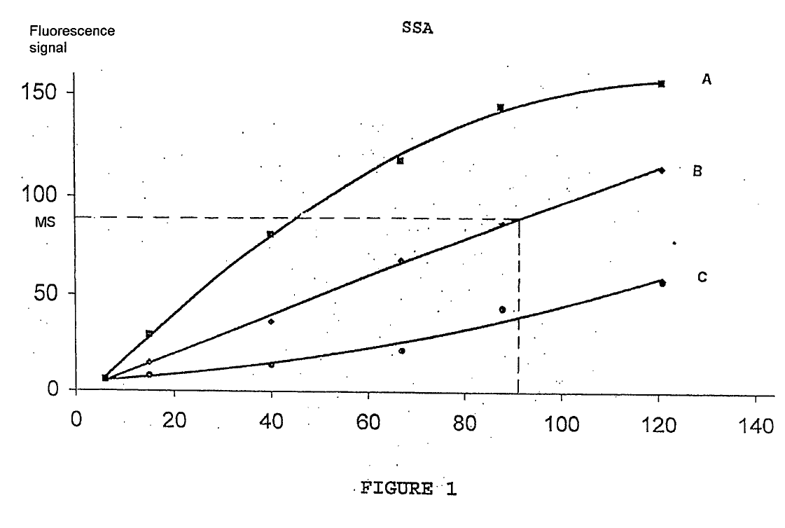

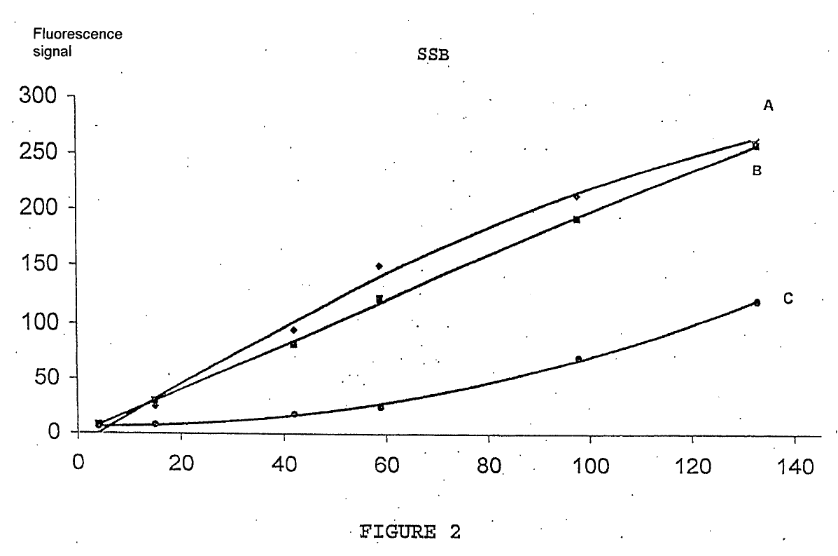

FIGS. 1 and 2 give, by way of example, three dose/response curves obtained for two types of ligands (SSA and SSB, respectively), expressing the fluorescence signal emitted (y-axis) as a function of the concentration of homologous compound in biological units (X-axis). These curves make it possible to select the smallest amount of ligand corresponding to a significant response signal, over a measurement range of 0 to 150 biological units, approximately.

For each system, five samples were selected as a function of their titer in biological units, determined by ELISA (ENA-LISA kit, sold by the applicant). The concentration of homologous compound, the sample size and the dilution are the same for all five samples.

As regards the SSA system (FIG. 1), the three curves represented under A, B, and C correspond, respectively, to concentrations of SSA antigens of 300, 150 and 75 μg/ml of particles. The curve selected for the SSA system is curve B, corresponding to a concentration of 150 μg of SSA antigen per ml of particles. The upper concentration of 300 μg (curve A) results in a saturation of the antigen-antibody reaction for the high values. The lower concentration of 75 μg (curve C) does not make it possible to obtain a sufficiently high response, hence a potential inaccurary in the results.

As regards the SSB system, the three curves represented under A′, B′ and C′ correspond, respectively, to concentrations of SSB antigens of 100, 50 and 25 μg/ml of particles. The curve selected for the SSB system is curve B′, corresponding to 50 μg of SSB antigen per ml of particles. The upper concentration of 100 μg (curve A′) resulting in a virtually identical curve is not selected. The lower concentration of 25 μg (curve C′) does not make it possible to obtain a sufficiently high response, in particular in the low values.

b) Calibration

A single calibration standard is prepared. In this example, the category of particles 1 attached to the SSA antigen is chosen. The calibration standard, i.e. the homologous compound, is here a solution, diluted in phosphate buffer, pH 7.4, of purified human antibodies directed against the SSA antigen.

The correspondence table for this calibration standard is as follows:

| | | | Sm/ | | | | |
|---|---|---|---|---|---|---|---|
| SSA | SSB | Sm | NRP | Scl70 | Jo1 | dsDNA | Centro. |
| 90 BU | 60 BU | 180 | 150 BU | 145 BU | 120 BU | 90 BU | 60 BU |
| | | BU | | | | | |

This table was obtained as indicated above.

c) Measurement of the Signal Emitted by the Calibration Standard

For the particles 1, sensitized with the SSA antigen, reacting with the calibration standard, a mean signal (MS), tested at least in duplicate, corresponding to 80, for example, is obtained by flow cytometry.

d) Determination of the Correction Factors

In order to determine the correction factors to be attributed to each specificity, the concentrations above, expressed in biological units in the correspondence table, are divided by the value of the signal measured for the calibration standard, i.e. the value obtained with the particles 1 sensitized with the SSA antigen (80).

The following correction factors are obtained:

| SSA | SSB | Sm | Sm/NRP | Scl70 | Jo1 | dsDNA | Centro. |
|---|---|---|---|---|---|---|---|
| 1.12 | 0.75 | 2.25 | 1.87 | 1.81 | 1.5 | 1.12 | 0.75 |

These correction factors will be used during the assaying of analytes in biological samples. Thus, if the SSB analyte is assayed in a sample by means of the immunoreagent having previously given a signal of 80 with the calibration standard, and a signal corresponding to 120 is obtained, the titer of the SSB analyte may be determined by multiplying the value of this signal by the correction factor associated with the SSB analyte (here 0.75). The SSB titer in the sample will therefore be, in this example, equal to 0.75×120=90 BU.

### EXAMPLE 2

### Simultaneous Assaying of Antineutrophil Cytoplasmic Antibodies (ANCA) Directed Against the Following Antigens: Myeloperoxidase (MPO) and Proteinase 3 (PR3)

a) Preparation of the Immunoreagent.

In this example, only two different colored particles are sensitized separately with the two antigens MPO and PR3, in an identical manner to example 1.

The immunoreagent then consists of a mixture of an aliquot of 500 μl of each category of colored particles. It is stored at +4° C. until use.

b) Calibration.

A single calibration standard is used for the calibration, according to the same principle of the invention. In this example, the category of particles attached to the MPO antigen is chosen. The calibration standard, i.e. the homologous compound, is here a solution, diluted in phosphate buffer, pH 7.4, of purified human antibodies directed against the MPO antigen. The protocol is the same as that used in example 1.

The correspondence table for this calibration standard is as follows:

| MPO | PR3 |
|---|---|
| 140 BU | 250 BU |

c) Measurement of the Signal Emitted by the Calibration Standard

The same protocol as in example 1 is used to determine said signal. The mean signal (MS) obtained for the calibration standard (tested in duplicate) reacting with the MPO particles corresponds, for example, to 150.

d) Determination of the Correction Factors

In order to determine the correction factors to attribute to each specificity, the above concentrations, expressed in biological units, are divided by the value of the signal for the calibration standard, i.e. the particles 1 sensitized with the MPO antigen (S=150).

The following correction factors are obtained:

| MPO | PR3 |
|---|---|
| 0.93 | 1.66 |

In a manner similar to example 1, if the PR3 analyte is assayed in a sample by means of the immunoreagent having previously given a signal of 150 with the calibration standard, and a signal corresponding to 110 is obtained, the titer for the PR3 analyte may be determined by multiplying the value of this signal by the correction factor associated with the PR3 analyte (here 1.66). The PR3 titer in the sample will therefore be, in this example, equal to 1.66×110=183 BU.

### EXAMPLE 3

### Evaluation of the Reagents Prepared in Examples 1 and 2 and Comparison with Reference Methods

In order to evaluate the immunoreagents prepared in examples 1 and 2 and to compare their results with those of reference methods of the ELISA type, the specificity and the sensitivity of said reagents were studied.

a) Determination of Specificity

The specificity was evaluated using 50 serum samples derived from blood donors and 34 samples selected for their potential biological interferences (hypergammaglobulinemia, monoclonal gammopathies, other autoantibodies, plasma samples, etc.). These samples constitute group 1.

b) Determination of Sensitivity.

The sensitivity was evaluated using serum samples, selected for each multiparametric assay, which were clinically characterized and derived from routine analysis in an immunology laboratory (Tenon Hospital, Paris, France). These samples constitute group 2, namely 57 and 35 samples, respectively, for the detection of ANA and of ANCA.

c) Protocol for Evaluating the Specificity and the Sensitivity of the Samples:

This protocol consists in taking 50 μl of the immunoreagent prepared in example 1 and mixing it, firstly, with 100 μl of calibration standard (deposited in duplicate) and, secondly, with 100 μl of samples prediluted 1/200 in a phosphate buffer (PBS type, pH 7.4). The depositing and mixing are carried out in 96-well microplates having a 1.2 μm filtering membrane at their base. One well is reserved for the mixture of the immunoreagent with the buffer alone, in order to constitute the “reagent blank”, making it possible to evaluate the signal associated with each category of particles, which will then be subtracted systematically from the signal obtained for the other wells.

The subsequent steps consist in:
incubating for 30 minutes at ambient temperature,
washing twice, with filtration through the filtering membrane of the microplate,
resuspending the medium in 100 μl of labeling reagent consisting of a goat antibody which reacts specifically with human G immunoglobulins and which is conjugated to phycoerythrin. Its concentration is suitable for all the “ligand-homologous compound” systems to be detected simultaneously,
incubating for 30 minutes, and
analyzing by flow cytometry.

That being so, at least 200 particles of each category are analyzed for 15 to 25 seconds according to the number of categories simultaneously present. During this time, the computer system classifies each of the particles according to its color and then determines the mean fluorescence emitted by the conjugate for each antibody specificity.

d) Comparative Methods Used.

The results obtained with the reagents prepared in examples 1 and 2 were compared with those derived from using commercial assay kits (Biomedical Diagnostics, Marne-La-Vallée, France): ELISA kits (DNA-LISA, ENA-LISA, MPO-LISA and PR3-LISA) and substrate Hep2000 for the determination by indirect immunofluorescence of the centromere.

Results

Evaluation of Specificity (Samples of Group 1).

detection of ANA: all the samples of group 1 were found to be negative by the multiparametric assay using the reagents prepared in example 1 and by the protocol using an ELISA kit.

detection of ANCA: all the samples of group 1 were found to be negative by the multiparametric assay using the reagents prepared in example 2; there was only one result which was not in agreement using an ELISA kit: it was a false positive for the determination of the PR3 antigenic specificity, developed by a sample derived from a patient exhibiting IgG hypergammaglobulinemia.

Evaluation of Sensitivity (Samples of Group 2)

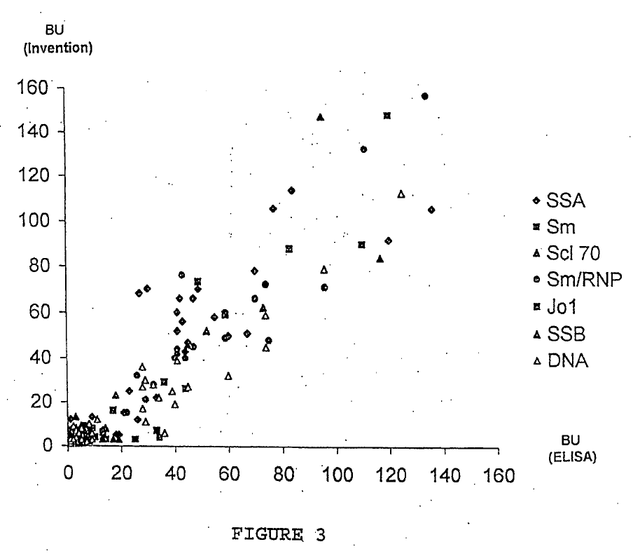

detection of ANA: it was carried out on 57 samples clinically characterized for the detection of ANA and derived from group 2. A 98.7% agreement was obtained on 399 assays carried out simultaneously with the multiparametric assay using the reagents prepared in example 1 and with an ELISA kit. FIG. 3 in the appendix shows the comparative results obtained, expressed in biological units (BU), for the multiparametric assay (Y-axis) compared with the individual ELISA assay (X-axis). The correlation coefficients are between 0.92 and 0.97 for the ANA specificities. For the centromere, complete agreement was obtained with indirect immunofluorescence (10 positive samples and 47 negative samples). The indirect immunofluorescence method consists of a semi-quantitative method using transfected Hep-2 human tumor cells as substrate. The autoantibodies bound to the substrate are revealed by means of an anti-human immunoglobulin conjugate labeled with fluorescein isothiocyanate. The presence of anti-centromere antibodies results in a punctate fluorescence on interphase nucleii. Reading is carried out under a fluorescence microscope, determining the final dilution of the sample where this fluorescence remains visible.

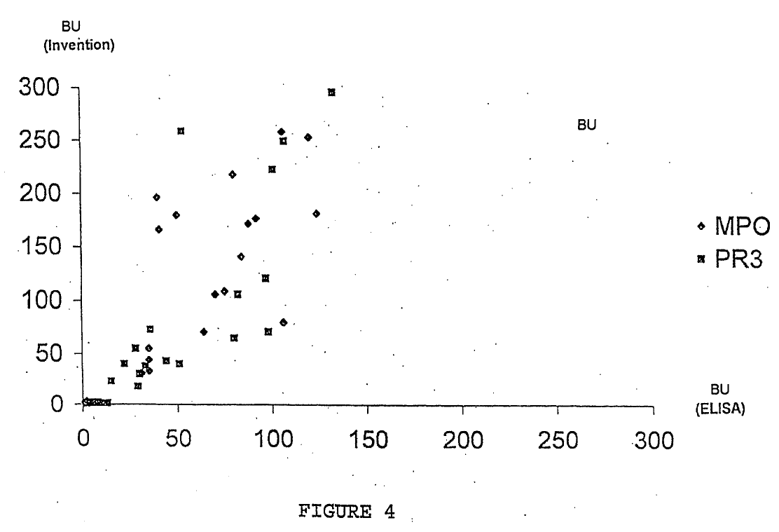

detection of ANCA: it was carried out on 35 samples clinically characterized for the detection of ANCA and derived from group 2. A 97.1% agreement was obtained on 70 assays carried out simultaneously with the multiparametric assay using the reagents prepared in example 2 and with an ELISA kit. FIG. 4 in the appendix shows the comparative results obtained, expressed in biological units (BU), for the multiparametric assay (Y-axis) compared with the individual ELISA assay (X-axis). The correlation coefficients are betweeen 0.87 and 0.85 for the ANCA specificities. These coefficients, which are lower than those for the detection of ANA, are due to the fact that the positive values are highly amplified in the case of the multiparametric assay.

1-19. (canceled)

20. A method for preparing an immunoreagent which goes into making up a calibration system applied to the assaying of multiple analytes in the same biological sample, said method using various categories of particles, each category of particles being sensitized by association with a ligand specific for one of the analytes to be assayed, said method characterized in that it comprises the steps which consist in:
a) determining, for each category of sensitized particles and for each of n given amounts of ligand associated with said particles, the curve of response as a function of the concentration of homologous compound, over a range of concentrations corresponding to the known measurement range of the analyte to be assayed;
b) selecting, for each category of sensitized particles, the curve corresponding to the smallest amount of ligand which gives a significant response signal and which is compatible with the use of a sample dilution and of a labeling reagent, which are common to all the analytes simultaneously assayed;
c) evaluating, for each category of particles sensitized, according to the curve selected in step b), the mean signal corresponding to the signal associated with a point characteristic of each of said curves, thus obtaining as many mean signals as there are categories of particles;
d) adjusting, where appropriate, the amounts of ligand associated with each category of particles such that all the mean signals evaluated in step c) are within a ratio of 1 to 5, and
e) mixing, in an appropriate solvent, the various categories of sensitized particles which correspond to the criterion of step d).

21. The method as claimed in claim 20, wherein, in step a), the amounts of ligand used vary by steps of 2 to 4.

22. The method as claimed in claim 20, wherein the sensitization of said particles with said ligands is carried out by covalence, by means of a biologically and/or chemically reactive intermediate molecular layer, or by using an affinity-based interaction system.

23. The method as claimed in claim 20, wherein the concentration of homologous compound is expressed in biological units over a range which is identical for all the analytes.

24. The method as claimed in claim 1, wherein the solvent used in step e) is suitable for and common to all the ligands.

25. The method as claimed in claim 20, wherein said ligands consist of antigens and/or of antibodies.

26. An immunoreagent intended for the assaying of multiple analytes in biological samples, said reagent comprising a solvent and, mixed in with said solvent, various categories of particles, each of which is sensitized by association with a given amount of a ligand specific for one of the analytes to be assayed, wherein, for each of the categories of particles, and for a given concentration of compound homologous to the ligand, as expressed in biological units, said given amount of ligand results in a signal referred to as “mean signal”, which is within a ratio of 1 to 5 with the mean signals obtained for the other categories of particles.

27. A kit for assaying multiple analytes in biological samples using various categories of particles, each category of particles being sensitized with a ligand specific for one of the analytes to be assayed, characterized in that it comprises:
i) an immunoreagent derived from the method as claimed in claim 20,
ii) at least one calibration standard consisting of a single homologous compound which reacts with one of the categories of particles which goes into making up the immunoreagent,
iii) a table of correspondence between the concentration, expressed in biological units, of the homologous compound constituting the calibration standard and that of each of the compounds homologous to the other ligands attached to the other categories of particles which go into making up the immunoreagent, and
iv) a labeling reagent.\

28. The kit as claimed in claim 27, wherein said calibration standard comprises a homologous compound which reacts directly or indirectly with the ligand attached to the sensitized particle.

29. The kit as claimed in claim 27, wherein said homologous compound is of the same origin as the analyte to be assayed.

30. The kit as claimed in claim 27, wherein said homologous compound and the analyte to be assayed are of different origins.

31. The kit as claimed in claim 27, wherein said labeling reagent consists of an immunocompound capable of quantifying the reaction between the analytes and the immunoreagent, said immunocompound being coupled to a label which is preferably a fluorochrome.

32. The kit as claimed in claim 27, wherein said labeling reagent consists of an immunocompound capable of quantifying the reaction between the analytes and the immunoreagent, said immunocompound being coupled to a label which is preferably a fluorochrome, and said labeling reagent reacting with the homologous compound or analyte to be assayed/ligand complex, in which case the assaying is direct, or with the uncomplexed ligand, in which case the assaying is indirect.

33. A method for using the kit as claimed in claim 27, characterized in that it consists in measuring the signals resulting from the interaction between the immunoreagent and, firstly, the biological sample, and secondly, the calibration standard, and in determining and applying, to the various resulting signals, a correction factor so as to obtain the titration, expressed in biological units, of each analyte of the sample, said correction factor being the ratio between the signal obtained for the calibration standard and the concentrations derived from the correspondence table.

34. The method for using the kit as claimed in claim 27, characterized in that it comprises the steps which consist in:
a) incubating, firstly, the biological sample and, secondly, the calibration standard with a predetermined amount of immunoreagent;
b) adding the labeling reagent;
c) measuring, by flow cytometry, the signals emitted, firstly, by the calibration standard and secondly, by the sample;
d) determining, for each category of particles sensitized, a correction factor which corresponds to the ratio between the concentration in biological units of each analyte to be assayed as given by the correspondence table and the signal measured in step c) for the calibration standard;
e) multiplying the signal emitted by each category of particles and measured in step c) by said correction factor, calculated for each analyte, so as to deduce therefrom the concentration in biological units of each analyte in the sample tested.

35. The method for using the kit as claimed in claim 27 in the simultaneous and direct quantification of antibodies having different antigenic specificities, characterized in that it comprises the steps of:
a) providing a biological sample in which said analytes to be assayed consist of antibodies;
b) providing the said immunoreagent in which the said ligands consist of antigens;
c) providing the said labeling agent which consists of one or more second antibodies labeled with a fluorochrome which react specifically with the antibodies intended to be assayed;
d) incubating, firstly, the said biological sample and, secondly, the said calibration standard with a predetermined amount of the said immunoreagent;
e) adding the said labeling reagent;
f) measuring, by flow cytometry, the signals emitted, firstly, by the said calibration standard and secondly, by the said sample;
g) determining, for each category of particles sensitized, a correction factor which corresponds to the ratio between the concentration in biological units of each analyte to be assayed as given by the correspondence table and the signal measured in step f) for the calibration standard;
e) multiplying the signal emitted by each category of particles and measured in step f) by said correction factor, calculated for each analyte, so as to deduce therefrom the concentration in biological units of each analyte in the sample tested.

36. The method for using the kit as claimed in claim 27 in the simultaneous and direct quantification of various antigens, characterized in that it comprises the steps of:
a) providing a biological sample in which said analytes to be assayed consist of antigens;
b) providing the said immunoreagent in which the said ligands consist of antibodies;
c) providing the said labeling agent which consists of a mixture of second antibodies labeled with a fluorochrome which react specifically with the antigens which are intended to be assayed;
d) incubating, firstly, the said biological sample and, secondly, the said calibration standard with a predetermined amount of the said immunoreagent;
e) adding the said labeling reagent;
f) measuring, by flow cytometry, the signals emitted, firstly, by the said calibration standard and secondly, by the said sample;
g) determining, for each category of particles sensitized, a correction factor which corresponds to the ratio between the concentration in biological units of each analyte to be assayed as given by the correspondence table and the signal measured in step f) for the calibration standard;
e) multiplying the signal emitted by each category of particles and measured in step f) by said correction factor, calculated for each analyte, so as to deduce therefrom the concentration in biological units of each analyte in the sample tested.

37. The method for using the kit as claimed in claim 27 in the simultaneous and indirect quantification of various antigens, characterized in that it comprises the steps of:
a) providing a biological sample in which said analytes to be assayed consist of antigens;
b) providing the said immunoreagent in which the said ligands consist of antibodies;
c) providing the said labeling agent which consists of a mixture of antigens labeled with a fluorochrome which compete with the analytes to be assayed for forming complexes with the ligands;
d) incubating, firstly, the said biological sample and, secondly, the said calibration standard with a predetermined amount of the said immunoreagent;
e) adding the said labeling reagent;
f) measuring, by flow cytometry, the signals emitted, firstly, by the said calibration standard and secondly, by the said sample;
g) determining, for each category of particles sensitized, a correction factor which corresponds to the ratio between the concentration in biological units of each analyte to be assayed as given by the correspondence table and the signal measured in step f) for the calibration standard;
e) multiplying the signal emitted by each category of particles and measured in step f) by said correction factor, calculated for each analyte, so as to deduce therefrom the concentration in biological units of each analyte in the sample tested.

38. The method for using the kit as claimed in claim 27 in the simultaneous and indirect quantification of various antibodies, characterized in that it comprises the steps of:
a) providing a biological sample in which said analytes to be assayed consist of antibodies;
b) providing the said immunoreagent in which the said ligands consist of antigens;
c) providing the said labeling agent which consists of a mixture of antibodies labeled with a fluorochrome which compete with the analytes to be assayed for forming complexes with the ligands;
d) incubating, firstly, the said biological sample and, secondly, the said calibration standard with a predetermined amount of the said immunoreagent;
e) adding the said labeling reagent;
f) measuring, by flow cytometry, the signals emitted, firstly, by the said calibration standard and secondly, by the said sample;
g) determining, for each category of particles sensitized, a correction factor which corresponds to the ratio between the concentration in biological units of each analyte to be assayed as given by the correspondence table and the signal measured in step f) for the calibration standard;
e) multiplying the signal emitted by each category of particles and measured in step f) by said correction factor, calculated for each analyte, so as to deduce therefrom the concentration in biological units of each analyte in the sample tested.
